(19) United States
(12) Patent Application Publication
(10) Pub. No.: US 2022/0011307 A1
(43) Pub. Date: Jan. 13, 2022

(54) DISK ELISA FOR QUANTITATIVE ANALYSIS

(71) Applicants: Eugene PERGAMENT, Chicago, IL (US); Rajasingam S. JEYENDRAN, Lisle, IL (US); PERJEY LLC, Chicago, IL (US)

(72) Inventors: Eugene PERGAMENT, Chicago, IL (US); Rajasingam S. JEYENDRAN, Lisle, IL (US)

(21) Appl. No.: 17/295,673

(22) PCT Filed: Dec. 3, 2019
(86) PCT No.: PCT/US2019/064180

§ 371 Date: May 20, 2021

Related U.S. Application Data
(60) Provisional application No. 62/774,589, filed on Dec. 3, 2018.

(51) Int. Cl. G01N 33/543; B01L 3/00

(52) U.S. Cl. CPC G01N 33/54391; B01L 2300/0803; B01L 2300/126; B01L 3/5023

(57) ABSTRACT

The present invention provides an Enzyme-Linked Immunosorbent Assay (ELISA) method for detecting and quantifying analytes. The present invention is advantageous over conventional methods, because the detection limit is not constrained by the sample volume or the length of time needed to perform quantitative ELISA.

### CROSS-REFERENCE TO RELATED APPLICATIONS

This application claims the benefit of priority under 35 U.S.C. § 119(e) to U.S. Provisional Patent Application No. 62/774,589, filed Dec. 3, 2018, the contents of which are incorporated by reference herein in their entirety.

### BACKGROUND OF THE INVENTION

Enzyme-Linked Immunosorbent Assay (ELISA) is a plate-based assay technique designed for detecting and quantifying antigens such as peptides, proteins, antibodies, and hormones. The key step in ELISA is the immobilization of an antigen. The antigen (or antibody) is generally immobilized directly or indirectly on a solid surface such as the polystyrene wall of a 96-well plate, which passively binds antigens and antibodies. The sample containing the antigen is placed inside the well and the antigen is captured either directly or indirectly via a capture antibody by gentle shaking, thereby enabling the antigen to encounter the capture antibody, which is further identified with a labeled detection antibody. This process takes time, since bringing the antigen and antibody together is a matter of chance. For example, the commercially available human hCG ELISA kit (www.thermoscientifc.com/elisa) needs more than 4 hours to complete the assay. Clinically, the test procedure is complex, time consuming, requires expensive equipment, trained technicians, and the need to satisfy CLIA requirements.

The Lateral Flow Immunoassay (LFIA) is a modification of the basic ELISA procedure and utilizes a nitrocellulose membrane strip. This membrane actively binds antibodies. The antigen and labeled detection antibody are allowed to migrate by capillary action towards an immobilized capture antibody on the strip. This takes about 10 minutes and generally yields whether the analyte in question is present in the tested sample. (See, for example, the Human Chorionic Gonadotropin (HCG) Test Kit, available through ARTRON LABORATORIES.) Clinically, the test procedure is simple to perform, requires minimal equipment, takes only minutes to perform, does not need trained technicians, and is usually a point of care test; however, LFIA is very difficult to develop.

There is a need in the art to develop an ELISA method that yields results at the point of care to facilitate the caregiver counseling to the client appropriately. Forcing the sample containing the antigen through a nitrocellulose membrane by gravitational filtration, centrifugation, or suction can reduce the length of time needed for the ELISA procedure to be about 30 minutes or less.

Both ELISA and LFIA methods can only use a small volume, about 100 μl or less, of the sample containing the antigen to be assayed. Due to the limitation posed by restricted volumes, it is difficult and sometime impossible to analyze and quantify the antigen in the sample containing low or extremely low concentration of antigens. Thus, there is a need in the art to develop a method that can be used to analyze samples with low to extremely low concentration/levels of antigen or any other analyte.

### SUMMARY OF THE INVENTION

In one aspect, the invention is a method for detecting and quantifying an analyte. The method includes immobilizing a first binding agent on a nitrocellulose membrane; placing the nitrocellulose membrane in either a spin column or a filter assembly; adding an analyte onto the nitrocellulose membrane; allowing the analyte to incubate with the first binding agent immobilized on the nitrocellulose membrane; adding a second binding agent onto the nitrocellulose membrane column; adding a wash buffer to the spin column; centrifuging the spin column for eliminating any excess and/or unbound first binding agent, analyte and/or second binding agent; adding a reagent (enzyme substrate) that interacts with the second binding agent to form a reporter species; and, quantifying the analyte by quantifying the reporter species with the help of a suitable detector.

In another aspect, the invention provides a kit for performing the method of the invention, wherein the kit comprises: the nitrocellulose membrane, the spin column or the filter assembly, the wash buffer, the first binding agent, the second binding agent, the reagent; and, an instruction manual for using the kit. In certain embodiments, the kit further comprises a competitive binding agent.

In certain embodiments, the analyte is an antigen. In certain embodiments, the antigen is selected from the group consisting of peptides, proteins, antibodies and hormones. In certain embodiments, the hormones are peptide or steroids or derivatives thereof.

In certain embodiments, the first binding agent is an antibody. In certain embodiments, the second binding agent is a conjugated antibody.

In certain embodiments, the conjugated antibody is selected from the group consisting of an antibody labelled with an enzyme such as horseradish peroxidase or alkaline phosphatase.

In certain embodiments, the enzyme substrate is selected from the group consisting of 3,3′,5,5′-Tetramethylbenzidine (TMB), 3,3′-Diaminobenzidine (DAB), 2,2′-azino-bis(3-ethylbenzothiazoline-6-sulphonic acid (ABTS) and luminol.

In certain embodiments, the reporter species is from the group consisting of a fluorescer, an enzyme, and a chemiluminescer.

In certain embodiments, the nitrocellulose membrane is disc shaped. In certain embodiments, the disc-diameter ranges from 0.5 mm to 20 mm.

In certain embodiments, the incubation temperature ranges from about 20° C. to about 40° C. In certain embodiments, the incubation time ranges from about 0.5 minutes to about 60 minutes.

In certain embodiments, the suitable detector is the one that measures the signal for enzyme activity, luminescence, and/or light absorbance.

In certain embodiments, a syringe is used in lieu of the spin column. In certain embodiments, the syringe is a 3 ml syringe.

In certain embodiments, a first type of filter assembly comprises an upper portion (male); a lower portion (female) comprising an port to allow for eliminating buffer, excess and/or unbound first binding agent, analyte, and/or second binding agent; wherein the discs interlock to form the assembly.

In certain embodiments, a second type of filter assembly comprising a first semicircular portion comprising two semicircular discs stacked over one another; a second semicircular portion comprising two semicircular discs stacked over one another, wherein, the first semicircle portion and the second semicircle portion interlock to form a filter assembly; a bottom port;

and a top port, wherein the bottom and the top ports can interchangeably act as inlet and outlet for movement of buffer, analyte, and binding agents through the filter. In another aspect, the invention provides a kit for performing the method of the invention, wherein the kit comprises: the nitrocellulose membrane; the spin column or a filter assembly, the wash buffer, the first binding agent; the second binding agent; the reagent (enzyme substrate); and, an instruction manual for using the kit.

### BRIEF DESCRIPTION OF THE DRAWINGS

For the purpose of illustrating the invention, certain embodiments of the invention are depicted in the drawings. However, the invention is not limited to the precise arrangements and instrumentalities of the embodiments depicted in the drawings

FIG. 1A is a table showing dose response based on human chorionic gonadotropin (hCG) concentrations vs luminescence intensity for Luminol substrate (n=9); (r²=0.992).

FIG. 1B is a graph showing dose response based on human chorionic gonadotropin (hCG) concentrations vs luminescence intensity for Luminol substrate.

FIG. 2A is a table showing dose response for very low hCG concentrations vs absorbance for TMB substrate (n=6); (r²=0.998).

FIG. 2B is a graph showing dose response for very low hCG concentrations vs absorbance for TMB substrate.

FIGS. 3A-3C show that the sample containing the antigen can be forced to flow through the nitrocellulose membrane by gravitational filtration, centrifugation and suction.

FIGS. 4A-4B show Enzyme-linked immunosorbent assay plate and lateral flow immunoassay.

FIGS. 5A -5B show spin column with 5 mm Disk and 3 ml disposable syringe with 10 mm Disk.

FIGS. 6A-6D show different views of a filter assembly. Top view (FIG. 6A); side views (FIGS. 6B. and 6C); perspective view (FIG. 6D).

FIG. 7 is a set of line diagrams showing the design of the lower portion (female) of the first type filter assembly.

FIG. 8 is a set of line diagrams showing the design of the upper portion (male) of the filter assembly.

FIG. 9 is a dose response curve for detecting human chorionic gonadotropin (hCG) using the first filter assembly.

FIG. 10A-10E show an alternate filter assembly, wherein a syringe or similar device is attached to both ends of the device, wherein the buffer, the analyte and the reagents can be filtered back and forth to improve the efficacy of the DISK ELISA. FIG. 10A is a cross-section view; FIG. 10B is perspective view ; FIG. 10C shows a locked filter device; FIG. 10D shows an unlocked filter device; FIG. 10E is a side-view of the filter assembly.

### DETAILED DESCRIPTION OF THE INVENTION

The present invention relates to methods for determining the presence and/or amount of analyte in a sample at the point of care.

### Definitions

As used herein, each of the following terms have the meaning associated with the term described in this section.

Unless defined otherwise, all technical and scientific terms used herein generally have the same meaning as commonly understood by a person of ordinary skill in the art to which this invention belongs. Generally, the nomenclature used herein and the laboratory procedures in cell culture, molecular genetics, biochemistry, biology, immunology, molecular biology, crystallography, and chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent in the context in which it is used. As used herein when referring to a measurable value such as an amount, a temporal duration, and the like, the term “about” is meant to encompass variations of ±20% or ±10%, ±5%, ±1%, or ±0.1% from the specified value, as such variations are appropriate to perform the disclosed methods.

The term “antibody,” as used herein, refers to an immunoglobulin molecule that specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including, virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized or can be derived from a biological sample. Such a biological sample can include, but is not limited to, a tissue sample, a tumor sample, a cell or a biological fluid.

The term “sample” used herein is any sample which is reasonably suspected of containing the analyte and/or antigen to be detected. The sample is typically an aqueous solution such as a body fluid from a host, for example, urine, whole blood, plasma, serum, saliva, semen, stool, sputum, cerebral spinal fluid, tears, mucus, or the like, spent embryo culture fluids but preferably is plasma or serum. The sample can be pretreated or prepared in any convenient medium which does not interfere with the assay.

The term “buffer” refers to any solution that may be used to achieve the desired pH and maintain the pH during the analysis. Illustrative buffers include but are not limited to borate, phosphate, carbonate, Tris, barbital and the like. The particular buffer employed is not critical to this invention but in individual assays, one buffer may be preferred over another.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a package or a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or downloaded from a website.

### Methods

In one aspect, the invention includes a method for detecting and quantifying an analyte; the method includes the steps of immobilizing a first binding agent on a 5 or 10 mm nitrocellulose membrane; placing the nitrocellulose membrane either in a spin column or a 3 ml disposable syringe or a filtering assembly; adding an analyte onto the nitrocellulose membrane; allowing the analyte to incubate with the first binding agent immobilized on the nitrocellulose membrane; adding a second binding agent onto the nitrocellulose membrane column, adding a wash buffer to the spin column or 3 ml disposable syringe; centrifuging the spin column or 3 ml disposable syringe for eliminating any excess and/or unbound first binding agent, analyte and/or second binding agent; transferring the nitrocellulose membrane from the spin column or 3 ml disposable syringe to another container for detection of the analyte; adding an enzyme substrate that interacts with the second binding agent to form a reporter species; and, quantifying the analyte by quantifying the reporter species with the help of a suitable detector.

The method of this invention is advantageous over conventionally used ELISA, because in conventional ELISA the detection limits, and hence the sensitivity of the assay, are constrained by restricted amount of sample volume that can be employed and the length of time needed. Contrary to conventional ELISA, in this method, the spin column or 3 ml disposable syringe attached to a nitrocellulose membrane can be loaded multiple times with a sample containing the antigen. In an exemplary embodiment, once the capacity of the spin column or 3 ml disposable syringe to hold the sample is reached, the column is centrifuged, the analyte is bound to nitrocellulose membrane, while remaining contents of the sample are centrifuged out and are discarded.

In certain embodiments, the analyte is an antigen and is a peptide, a protein, an antibody or a peptide or steroid hormone or a derivative thereof.

In certain embodiments, the first binding agent is an antibody that can bind to the analyte.

In certain embodiments, the analyte is incubated with the first binding agent at the temperature ranges from 1° C. to 40° C. In certain embodiments, the incubation temperature is about 1, 5, 10, 15, 20, 25, 30, 35, or about 40° C. In certain embodiments, the incubation time varies from 0.5 min to 60 min. In certain embodiments, the incubation time varies between about 0.5, 5, 10, 15, 20, 25, 30, 35, 40, 45, 50, 55, or about 60 min.

In certain embodiments, the second binding agent is an antibody that is a labelled or conjugated to another molecule including but not limited to an enzyme. Suitable labeling methods that can be used in the present invention include, without limitation, isotope labeling, chemical modification, enzyme conjugation, fluorescent dye labeling, luminescence and other labeling methods commonly known by those skilled in the art. In certain embodiments, the second binding agent includes, but is not limited to, a chemical molecule, a peptide molecule, a protein molecule, an RNA molecule, a DNA molecule, a traditional antibody, a fragment of a traditional antibody containing an antigen binding site, a recombinant antibody containing an antigen binding site, a protein which binds to an antigen, a bacterial cell, a viral particle, a cell, a particle, and a product comprising crosslinking any two or more of those listed above. In an exemplary embodiment, the second binding agent is a conjugated antibody and is selected from the group consisting of an antibody labelled with horseradish peroxidase or alkaline phosphatase.

In certain embodiments, the container in which the sample is analyzed is selected from the group consisting of a glass or polypropylene tube, or cuvettes made from, for example, polystyrene or any other suitable material.

In certain embodiments, the enzyme substrate interacts with the second binding agent to form a reporter species. For example, the substrate is selected from 3,3′,5,5′-Tetramethylbenzidine (TMB), 3,3′-Diaminobenzidine (DAB), 2,2′-azino-bis(3-ethylbenzothiazoline-6-sulphonic acid (ABTS) and luminol. In certain embodiments, the reporter species is a fluorescer, enzyme, chemiluminescer or photosensitizer and thus the amount of reporter species can be detected using enzyme activity, luminescence or light absorbance assays. In an exemplary embodiment, the reporter species is formed to effect the color change. The intensity of the color change is proportional to the amount of analyte in the sample.

In certain embodiments, a detection system such as a spectrophotometer, a fluorometer or a luminometer is employed to detect and quantify the reporter species.

In certain embodiments, the nitrocellulose membrane is disc shaped having a diameter ranging from about 0.5 mm to about 20 mm.

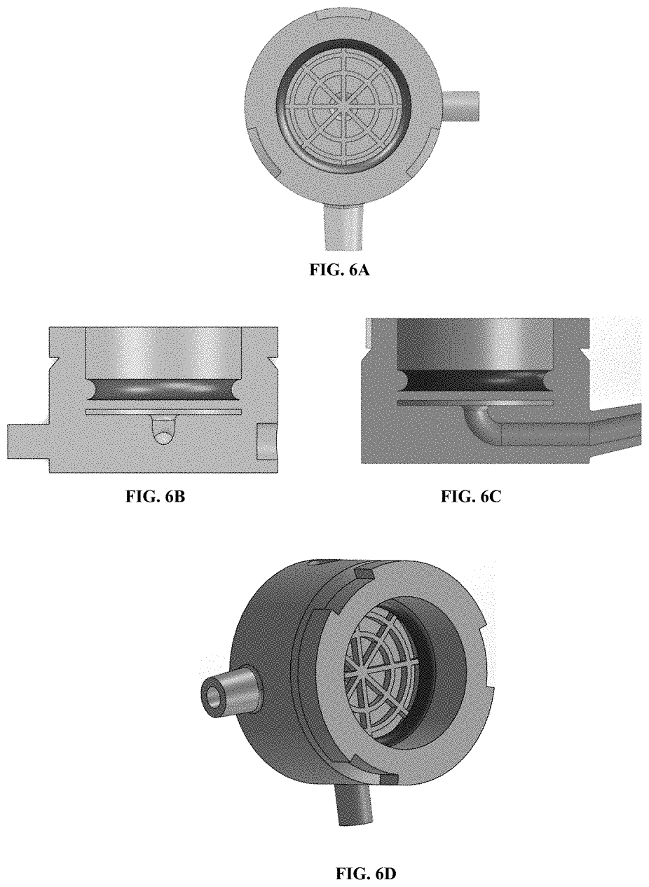

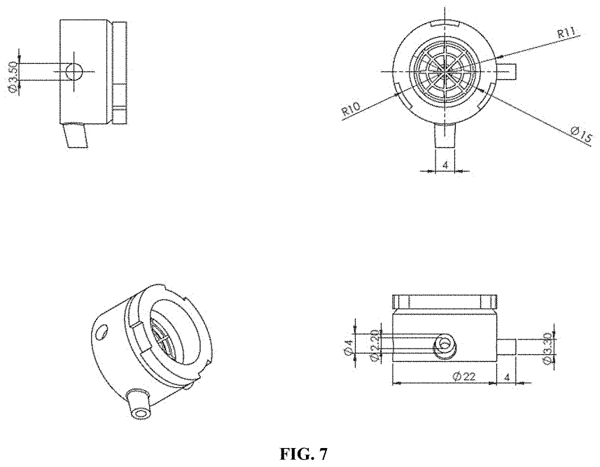

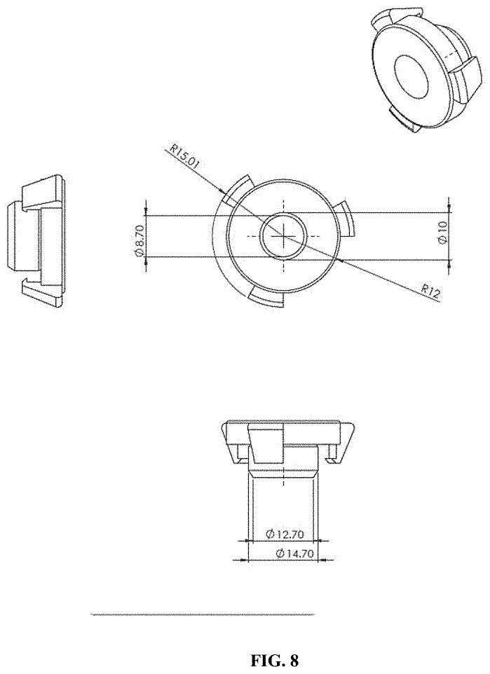

In certain embodiments, a first type of filtering assembly (FIGS. 6A-6D) includes an upper portion (male) and a lower portion (female) comprising an arm to allow for elimination of buffer, excess and/or unbound first binding agent, analyte, and/or second binding agent In certain embodiments, there are two ports projecting from the bottom portion (female) of the filtering assembly. One port is a solid post that may be used to insert into a matched depression in an adjacent identical filtering assembly, to allow a group of filter assemblies to be linked together to form a chain that can be manipulated simultaneously to process multiple samples or standards at once, including but not limited to, attaching each filter assembly's output port to a vacuum suction manifold. The second projecting port is the outlet port that allows wash buffer or other solutions to exit from the filter assembly.

In certain embodiments, the upper and the lower portions interlock to form the filter assembly. In one preferred embodiment, the upper and the lower portions are made from a polymer, for example, ABS Green, any organic polymer, mixed polymer or other material including, but not limited to stainless steel or other alloys deemed to generally be chemically nonreactive in the aqueous and organic chemical reactions that are intended to be used in this apparatus for the purposes described.

In certain embodiments, a second type of filter assembly includes a first semicircular portion comprising two semicircular discs stacked over one another; a second semicircular portion comprising two semicircular discs stacked over one another, wherein, the first semicircle portion and the second semicircle portion interlock to form a filter assembly; a bottom port; and, a top port, wherein the bottom and the top ports can interchangeably act as inlet and outlet for movement of buffer, analyte, and binding agents through the filter. In certain embodiments, the assembly further comprises a first syringe attached to a top portion of the assembly and, a second syringe attached to a bottom portion of the assembly to facilitate the back and forth movement of buffer, analyte and binding agents through the filter.

In certain embodiments, the buffer, the binding agents and the analyte are introduced into the filter assembly by means of a syringe and piston. In certain embodiments, the buffer, the binding agents, and the analyte are introduced into the filter assembly by hand-held manual or electronic pipetting device or disposable single use microcapillary droppers. In certain embodiments, the droppers are made from polymeric materials including but not limited to polypropylene or in some cases glass, including but not limited to borosilicate.

In certain embodiments, the procedure is completed in three steps within one container.

In certain embodiments, when the antigen is filtered through the filter assembly, it binds with the second binding agent on the glass fiber disc before binding with the first binding agent that is immobilized on the nitrocellulose membrane.

In certain embodiments, when the antigen is filtered through the filter assembly, it binds to the first binding agent before binding with the second binding agent.

In certain embodiments, the procedure comprises steps of: mixing the analyte with the second binding agent and placing the mixture on the nitrocellulose disk; adding a wash buffer after at least ten minutes of commencing the procedure; removing the wash buffer and any residual liquid; adding the color development reagent such as TMB or Luminol and any additional necessary reagents such as hydrogen peroxide; adding the substrate; transferring the reacted substrate into a different container appropriate for the type of signal detection instrument used; and detecting the concentration of the analyte using a suitable detector.

### KIT

In one aspect, the invention is a kit for using the assay method for detecting antibodies in a sample suspected of containing the antibodies. A typical kit of this invention comprises in a packaged combination of a nitrocellulose membrane, a spin column, a wash buffer, a first binding agent, a second binding agent, a substrate, positive and negative control, and an instruction manual for using the kit. In certain embodiments, the kit further comprises a competitive binding agent.

In certain embodiments, one or more of the contents of the kit can be provided in solution or as a dry powder, usually lyophilized, which on dissolution provides a solution having the appropriate concentrations for performing a method or assay in accordance with the present invention. In certain embodiments, the contents of the kit are provided in packaged combination, in the same or separate containers, so that the ratio of the reagents provides for substantial optimization of the method and assay. In certain embodiments, the reagents employed in the present invention can be provided in predetermined amounts. In certain embodiments, the kit instruction manual provides instruction on how to use the reagents and/or how to perform a particular assay, for example, in the form of a package insert.

### EXAMPLES

The invention is now described with reference to the following Examples. These Examples are provided for the purpose of illustration only, and the invention is not limited to these Examples, but rather encompasses all variations that are evident as a result of the teachings provided herein.

Without further description, it is believed that one of ordinary skill in the art can, using the preceding description and the following illustrative examples, practice the claimed methods of the present invention. The following working Examples therefore specifically point out the preferred embodiments of the present invention, and are not to be construed as limiting in any way the remainder of the disclosure.

### Example

### Validating that Nitrocellulose Membranes Can be Used Instead of Polystyrene Plates

To validate whether a nitrocellulose disk could be utilized instead of the polystyrene plates employed for the standard ELISA procedure, a pair of previously tested monoclonal capture and detection mouse antibodies against human Chorionic Gonadotropin (hCG) and 5 mm diameter disks were tested as follows:

Three 5 mm diameter nitrocellulose disks were incubated overnight with 2.5 μl of capture antibody. The disks were manually washed 5 times with wash buffer and dried on a filter paper. To one of the washed and dried disk 2.5 μl of 100% hCG was added to one disk, 2.5 μl of 10% hCG was added to the second disk and 2.5 μl of PBS (control) was added to the third disk respectively. All three disks were incubated at room temperature for 20 minutes and washed and dried as before. 2.5 μl of HRP labeled detection antibody was added to all three disks and incubated 5 minutes and washed and dried as before. To each disk 100 μl of Luminol: H₂O₂ (1:1, v/v) was added and luminescence intensity was read immediately using Spectramax L. A linear dose response for hCG concentration tested was obtained for 50 mIU/m1 (23.1×10⁶); 5 mIU/ml (3.31×10⁶) and 0 mIU/ml (0.50×10⁶) respectively confirming the use of nitrocellulose disk to validate the new procedure “DISK ELISA”.

### Example 2

### Validating “DISK ELISA” Procedure

To validate the “DISK ELISA” procedure, eighteen 5 mm diameter nitrocellulose disk were incubated for 3 hours with 2.5 μl of capture antibody and blocked with 5% bovine serum albumin overnight. Three disks were placed inside three different Econo spin column (www.epochbiolabs.com; Cat. No. 1920-250) and 5 μl of 255 mIU/ml, 5μl of 134 mIU/ml and 0 mIU/ml, respectively were added and incubated at room temperature for 10 minutes. To each disk, 2.5 μl of detection antibody conjugated to HRP was added and incubated at room temperature for 5 minutes. They were then washed 3 times with 1.5 ml of fast wash buffer, spun down on high for about 30 seconds on a Galaxy Mini Centrifuge (www.vwr.com). The disks were transferred from the spin tubes to glass tubes and 150 μl of Luminol: H₂O₂ (1:1, v/v) was added, incubated for 5 minutes in dark and luminescence intensity was read using Spectramax L.

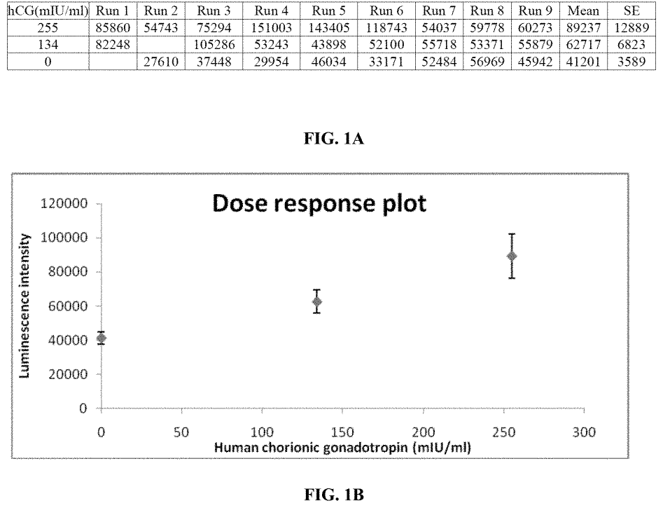

The experiment was repeated 9 different times and the results are as shown in FIGS. 1A-1B.

### Example 3

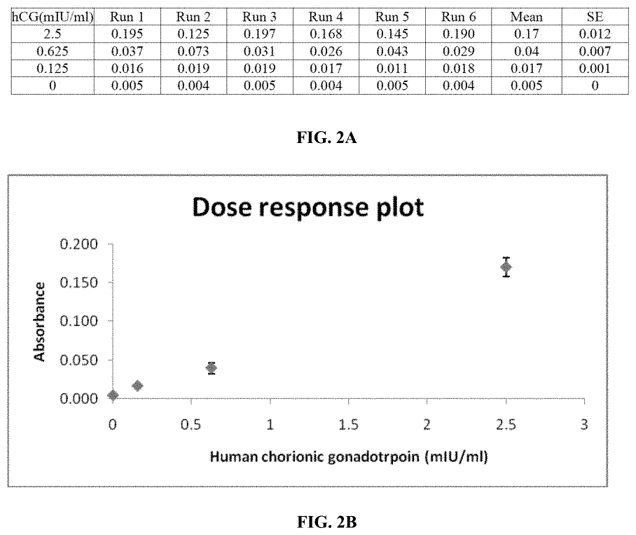

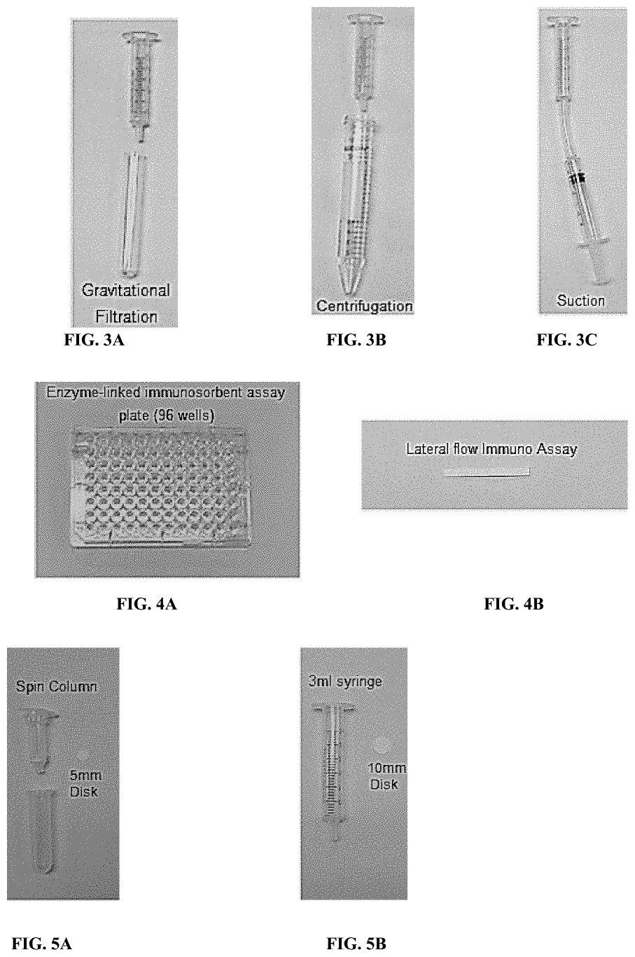

An experiment similar to that described in Example 2 was performed with 2.5 mIU/ml, 0.62 5mIU/ml, 0.125 mIU/m1 and 0 mIU/ml, except TMB (3,3′,5,5′-Tetramethylbenzidine) substrate was used as an end point signal-detection system. The experiment was repeated 6 different times and the results are as shown in FIGS. 2A-2B.

### Example 4

### Validating Use of Filter Assembly for “DISK ELISA”

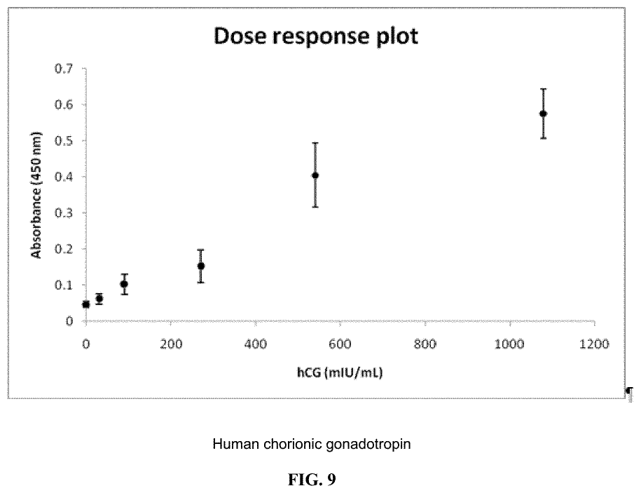

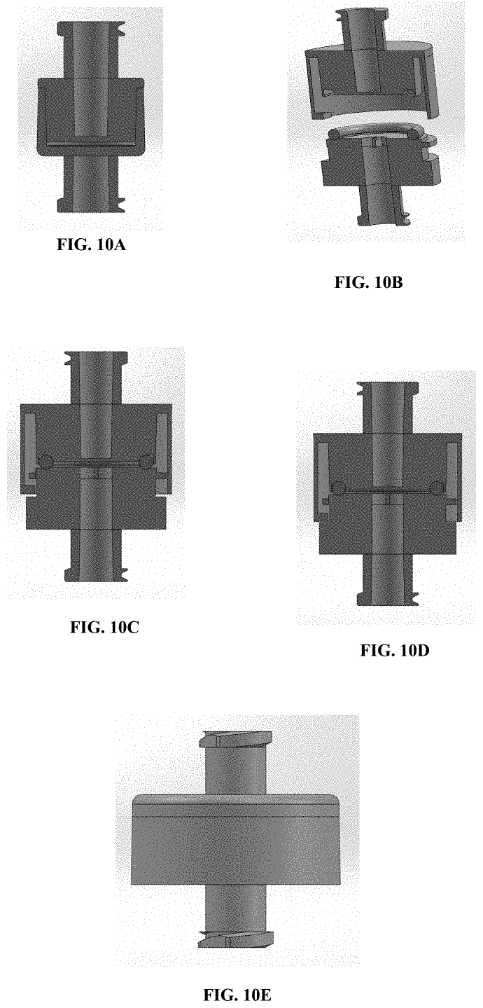

A filter assembly was used instead of the spin column for detecting hCG and the results are shown in FIG. 9.

### Other Embodiments

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. Although this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations

1. A method for detecting and quantifying an analyte, the said method comprising:
(a) immobilizing a first binding agent on a nitrocellulose membrane;
(b) adding an analyte onto the nitrocellulose membrane placed inside a spin column or a filter assembly;
(c) adding a second binding agent onto the nitrocellulose membrane column;
(d) adding a wash-buffer to the spin column;
(e) centrifuging the spin column for eliminating any excess and/or unbound first binding agent, analyte and/or second binding agent;
(f) adding a substrate that interacts with the second binding agent to form a reporter species; and
(g) quantifying the analyte by quantifying the reporter species with the help of a suitable detector.

2. The method of claim 1, wherein the analyte is an antigen.

3. The method of claim 2, wherein the antigen is selected from the group consisting of peptides, proteins, antibodies, and hormones.

4. The method of claim 1, wherein the first binding agent is an antibody.

5. The method of claim 1, wherein the second binding agent is a conjugated antibody.

6. The method of claim 5, wherein the conjugated antibody is an antibody labelled with horseradish peroxidase or an alkaline phosphatase.

7. The method of claim 1, wherein, after step (b), the analyte is allowed to incubate with the first binding agent.

8. The method of claim 1, wherein the substrate is selected from the group consisting of 3,3′,5,5′-Tetramethylbenzidine (TMB), 3,3′-Diaminobenzidine (DAB), 2,2′-azino-bis(3-ethylbenzothiazoline-6-sulphonic acid (ABTS) and luminol.

9. The method of claim 1, wherein the reporter species is from the group consisting of a fluorescer, an enzyme, and a chemiluminescer.

10. The method of claim 1, wherein the nitrocellulose membrane is disc shaped.

11. The method of claim 10, wherein the disc-diameter ranges from about 0.5 mm to about 20 mm.

12. The method of claim 7, wherein the incubation temperature ranges from about 1° C. to about 40° C.

13. The method of claim 7, wherein the incubation time ranges from about 0.5 minutes to about 60 minutes.

14. The method of claim 1, wherein the suitable detector is the one that measures, the signal for enzyme activity, luminescence, and/or light absorbance.

15. The method of claim 1, wherein a syringe is used in lieu of the spin column.

16. The method of claim 15, wherein the syringe is a 3 ml syringe.

17. The method of claim 1, wherein the filter assembly comprises
an upper portion (male);
a lower portion (female) comprising an arm to allow for eliminating buffer, excess and/or unbound first binding agent, analyte, and/or second binding agent;
wherein the discs interlock to form the assembly.

18. The method of claim 1, wherein the filter assembly comprises
a first semicircular portion comprising two semicircular discs stacked over one another;
a second semicircular portion comprising two semicircular discs stacked over one another; wherein, the first semicircle portion and the second semicircle portion interlock to form a filter assembly.
a bottom port;
a top port, wherein the bottom and the top ports can interchangeably act as inlet and outlet for movement of buffer, analyte, and binding agents through the filter.

19. The method of claim 18, wherein the assembly further comprises a first syringe attached to a top portion of the assembly and a second syringe attached to a bottom portion of the assembly to facilitate the back and forth movement of buffer, analyte and binding agents through the filter.

20. A kit for performing the method of claim 1, wherein the kit comprises:
the nitrocellulose membrane of claim 1;
the spin column or the filter assembly of claim 1,
the wash buffer of claim 1,
the first binding agent of claim 1;
the second binding agent of claim 1;
the reagent of claim 1; and,
an instruction manual for using the kit.

21. The kit of claim 20, wherein the kit further comprises a competitive binding agent.
